(19) United States
(12) Patent Application Publication
(10) Pub. No.: US 2024/0238108 A1
(43) Pub. Date: Jul. 18, 2024

(54) PROSTHESIS

(71) Applicants: Aidan Roche, Edinburgh (GB); Koalaa Limited, London (GB)

(72) Inventors: Aidan Roche, Edinburgh (GB); Nate Macabuag, London (GB); Gino La, London (GB)

(21) Appl. No.: 18/429,811

(22) Filed: Feb. 1, 2024

Related U.S. Application Data
(63) Continuation of application No. PCT/IB2022/056899, filed on Jul. 26, 2022.

(30) Foreign Application Priority Data
Aug. 2, 2021 (GB) 2111117.4

(51) Int. Cl. A61F 2/78; A61F 2/70; A61F 2/76

(52) U.S. Cl. CPC A61F 2/78; A61F 2002/701; A61F 2002/707; A61F 2002/7635

(57) ABSTRACT

The present invention relates to a prosthesis for an arm or a leg, comprising a flexible sleeve for receiving a residual limb, a constrictor on the flexible sleeve for tightening the sleeve around the residual limb, and a rigid mount secured to the sleeve and spaced axially along the sleeve from the constrictor for mounting a tool. A sensor is provided in the mount to measure a physical parameter associated with the function performed by the tool. The prosthesis further comprises an actuator connected to the constrictor and a control circuit acting on the actuator to vary the force tightening the sleeve around the residual limb in dependence upon the measured parameter.

### CROSS REFERENCE TO RELATED APPLICATIONS

Continuation of International Application No. PCT/IB2022/056899 filed on Jul. 26, 2022. Priority is claimed from British Application No. 2111117.4 filed on Aug. 2, 2021. Each of the foregoing applications are incorporated herein by reference in its entirety.

### STATEMENT CONCERNING FEDERALLY SPONSORED RESEARCH OR DEVELOPMENT

Not applicable.

### NAMES OF THE PARTIES TO A JOINT RESEARCH AGREEMENT

Not applicable.

### FIELD OF THE INVENTION

The present invention relates to a prosthesis to be fitted to a residual limb of a wearer.

### BACKGROUND

Prostheses for partial limbs are widely known and typically made of rigid materials or have, increasingly, very complex electronics that aim to provide an artificial limb (e.g. hand). Some prior art patents seek to protect a scaled down, soft sleeve prosthesis, such as GB 2570018, which discloses a prosthesis for an arm or a leg that includes a flexible sleeve for receiving a residual limb, a constrictor on the flexible sleeve for reducing the periphery of the flexible sleeve and tightening the sleeve around the residual limb, and a rigid mount secured to the sleeve and spaced axially along the sleeve from the constrictor for mounting a tool, such as a pen, an artificial hand or an electrical tool.

Increasingly, prior art prostheses are becoming complex, providing sophisticated electronics that interact with nerve impulses in the residual limb to trigger certain movements in a, for example, artificial (robotic) hand at the distal end of the prosthesis. These prostheses provide a high level of feedback for the wearer. However, the cost of such technology is prohibitively high for all but the wealthiest users.

By contrast, the scaled down, cost-effective prior art solutions lack any significant feedback for the wearer. It would be desirable, for example, for the wearer to know how much pressure they are applying to a knife, pencil or paintbrush held in a prosthetic arm. There are currently no solutions available to meet this need in a cost-effective manner. Thus, it is desirable to provide a prosthesis that is able to provide a degree of feedback to the wearer in response to a parameter detected by the prosthesis. For example, in response to the amount of pressure applied to a tool at the distal end of the prosthesis.

### SUMMARY OF THE INVENTION

With a view to assisting the wearer in controlling the tool mounted on the prosthesis, in the present invention a physical parameter associated with the function performed by the tool is measured by means of a sensor located either in the mount, or in a tool mounted on the prosthesis, and the tightening force applied by the constrictor to the flexible sleeve gripping the residual arm of the wearer is modified in dependence upon the measured parameter.

Thus, in accordance with a first aspect of the present invention, there is provided a prosthesis for an arm or a leg, comprising a flexible sleeve for receiving a residual limb, a constrictor on the flexible sleeve for tightening the sleeve around the residual limb, and a rigid mount secured to the sleeve and spaced axially along the sleeve from the constrictor for mounting a tool, wherein a sensor is provide in the mount to measure a physical parameter associated with the function performed by the tool and wherein the prosthesis further comprises an actuator connected to the constrictor and a control circuit acting on the actuator to vary the force tightening the sleeve around the residual limb in dependence upon the measured parameter.

In accordance with a second aspect, the invention provides a prosthesis for an arm or a leg, comprising a flexible sleeve for receiving a residual limb, a constrictor on the flexible sleeve for tightening the sleeve around the residual limb, a rigid mount secured to the sleeve and spaced axially along the sleeve from the constrictor and a mount for releasably attaching to the prosthesis a tool that incorporates a sensor for measuring a physical parameter associated with the function performed by the tool, wherein the prosthesis comprises an actuator connected to the constrictor and a control circuit communicating with the sensor by way of the mount and acting on the actuator to vary the force tightening the sleeve around the residual limb in dependence upon the measured parameter.

The nature of the measured parameter and the positioning and type of the sensor employed will depend on the tool and the function performed by the tool.

In one embodiment, the tool is a pen and the wearer is provided with feedback through the gripping force (degree of tightening) applied by the flexible sleeve to the residual limb to indicate the pressure being applied by the writing nib.

In such an embodiment, a pressure sensor or strain gauge may be provided in the mount.

Alternatively, a pressure sensor or strain gauge may be incorporated in the pen and its signal communicated in a suitable manner to the prosthesis. Such communication between the tool and the prosthesis may simply involve the provision of electrical contacts on the mount and the pen, or it may be carried out by a contactless link, such as an inductive loop, an electro-optical coupling, or wireless (e.g. Bluetooth®).

As earlier stated, the physical parameter providing haptic feedback to the wearer would depend on the type of tool attached secured to the mount of the prosthesis.

For example, if the tool is a motorized hand capable of applying a variable grip, the sensor may serve to indicate to the wearer the gripping force being applied. In the case of an electric tool, the sensor may indicate the load on the motor of the tool. With other tools, the sensed parameter may be a temperature.

The above are examples of tools for a prosthesis where the residual limb is an arm. However, the invention is also applicable to prostheses to be fitted to a leg. For example, if the tool is an artificial foot used by a wearer to drive a vehicle, the sensor may be used to indicate the force being applied to a pedal.

In some embodiments of the invention, the constrictor tightening the sleeve around the residual limb may be an electric motor having an output shaft about which there is wrapped an elongate flexible member connected to the flexible sleeve.

The elongate flexible member may be a band encircling the flexible sleeve and the residual limb. Alternatively, the flexible sleeve may have an axially extending slit or gusset and the flexible member may be threaded through eyelets in the edges of the sleeve defining the slit or gusset to act in a manner analogous to a boot lace.

A control circuit employed to vary the gripping force in dependence upon the magnitude of the measured physical parameter may include an encoder mounted on the output shaft of the motor acting on the flexible member, the encoder providing a feedback signal to enable the control circuit to rotate the output shaft of the motor to a pre-calibrated position corresponding to the value of the sensed parameter.

### BRIEF DESCRIPTION OF THE DRAWINGS

The invention will now be described further, by way of example, with reference to the accompanying drawings, in which:

FIG. 1 is a perspective view of a prosthesis,

FIG. 2a is a diagram illustrating the connection between components of the prosthesis in a first embodiment of the invention,

FIG. 2b is a diagram illustrating the connection between components of the prosthesis in an alternative embodiment of the invention, and

FIG. 3 is a schematic representation of an actuator.

### DETAILED DESCRIPTION

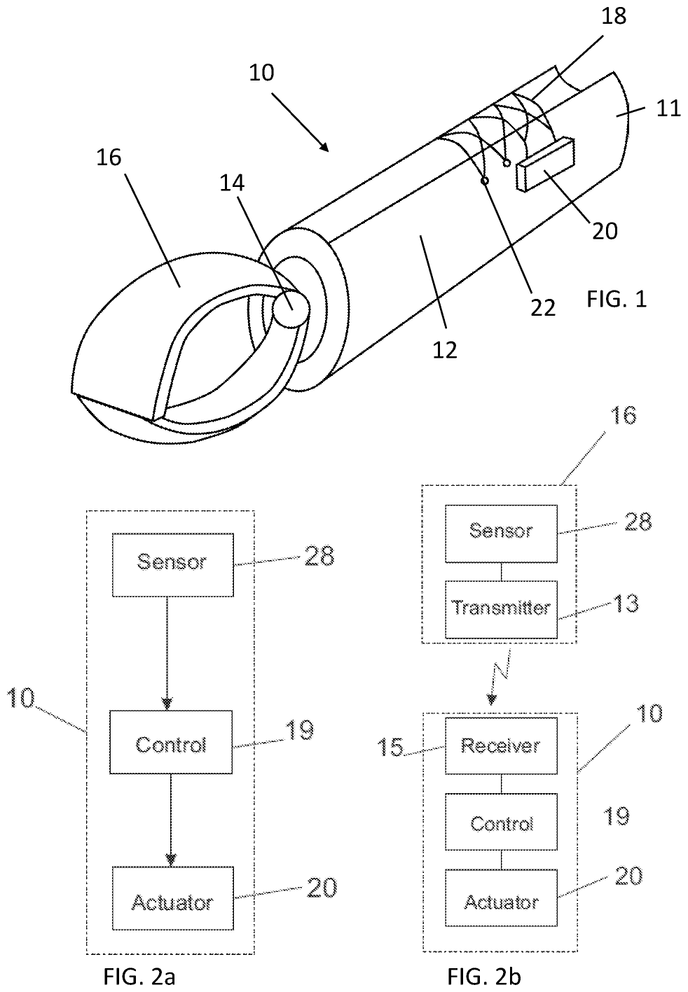

FIG. 1 shows a prosthesis 10 for an arm. The prosthesis 10 comprises a sleeve 12 for fitting over a residual limb (not shown). A rigid mount 14 for holding a tool 16 is fitted to the distal end of the sleeve 12. The mount 14 being an analogue of a hand. The proximal end 11 of the sleeve 12 is flexible and can be tightened around the arm of the user. To provide the wearer with feedback from the tool, the grip of the proximal end 11 of the prosthesis can be varied to represent a measured parameter. In the case of a pen, such a parameter may be the amount of force being applied by the nib of the pen. The wearer may then control the amount of force applied to the tool, based on the feedback. The proximal end 11 of the sleeve 12 that is to be tightened about the residual limb may be made of the same or of a different material from the rest of the sleeve 12.

The sleeve 12 is shown as one that tapers towards the distal end to resemble a natural limb that tapers from the elbow to the wrist. The sleeve 12 may however be of any shape to match the wearer's residual limb.

The word “flexible” as applied to the proximal end 11 of the sleeve 12 is intended to mean that the sleeve diameter can be modified to vary the grip on the residual limb of the wearer. It may, if desired, be additionally able to bend along its major axis to accommodate different shapes of limb. The sleeve 12 may be made of any material that allows the conditions above, such as a plastics sheeting or a fabric.

The cross section of the proximal end of the may be continuous, or it may have a gusset or a slit to allow its diameter to be changed. If a slit is present, a protective tongue may be provided beneath the slit for the comfort of the wearer.

The proximal end 11 of the sleeve 12 is tightened around the arm of the wearer by means of a constrictor 18 which is tensioned by an actuator 20. In some embodiments, the constrictor 18 may take the form shown in FIG. 1 of a lace passing through eyelets 22, the lace being tightened by the actuator 20 by pulling both ends of the lace. It may be that one end of the lace is fixed and therefore only the non-fixed end needs to be pulled to tighten the constrictor 18. Alternatively, the constrictor 18 may be a belt fixed at one point of the sleeve 12 and extending circumferentially around the sleeve to the actuator 20 so that the actuator can pull the belt tight.

To tighten and release the constrictor 18 around the arm of the wearer, a control circuit 19 is provided to control the current supply to the actuator 20 based on a measured parameter related to the function being performed by the tool 16.

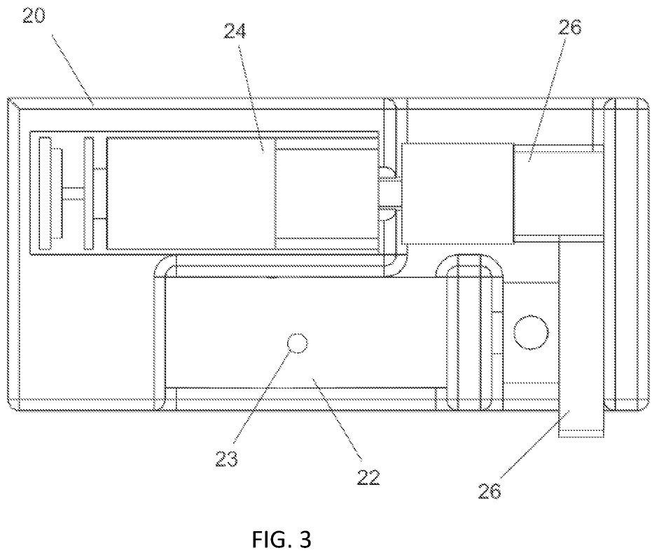

In an embodiment shown in FIG. 3, the constrictor (omitted for clarity) is wrapped around a shaft 22 in the housing of the actuator 20, and as the shaft 22 rotates it reels in the constrictor 18 causing it to tighten. The shaft 22 may have a hole 23 extending radially through it in order for the constrictor 18 to be tied down. The shaft 22 is rotated by a motor 24 and the output torque from the motor 24 is increased by means of intermeshing gears 26. Appropriate reduction, or step-down, gearing also facilitates a smaller and therefore lighter motor 24 to be fitted, thus reducing the mass of the prosthesis 10.

In an alternative embodiment, the actuator may comprise a rack and pinion mechanism, with the rack connected to the constrictor.

The constrictor 18 in the embodiment illustrated in FIG. 1 may serve additionally as the primary, or sole, means of securing the prosthesis 10 to the residual limb. This however need not be the case and it is possible for there to be provided an additional means for enabling the proximal end of the sleeve to grip the residual limb of the user.

The control circuit 19 (shown in FIGS. 2a and 2b) controls the operation of the motor 24 by processing data from a sensor 28 and then supplying instructions to the actuator 20 to tighten or slacken the constrictor 18 by a specific amount. The shaft 22 may be fitted with an encoder for monitoring its angular position to enable the position of the shaft 22 to be set by means of a closed control loop.

The sensor 28 may be incorporated into the mount 14 to allow use of any tool 16, e.g. a regular pen or pencil, without requiring the tool to me modified by incorporation of a sensor. This is shown diagrammatically in FIG. 2a. In alternative embodiments, shown in FIG. 2b, the sensor 28 is incorporated in a dedicated or modified tool 16, and a wired or wireless transmission link is used to communicate the output of the sensor 28 to the control circuit 19.

The mount 14 is attached to the end of the sleeve 12 to act as a hand. The attachment of the mount 14 is achieved by any suitable means. Interchangeable mounts 14 may be provided capable of performing different tasks (writing, gripping etc.).

The mount 14 itself may be of any shape that lends itself to the task to be completed and to the shape of the tool performing the task. For example, the mount may include a tube formed of a compressible foam into which pens of different diameter may be inserted. The tube may be inclined to the longitudinal axis of the sleeve 12 so that the reaction force on the nib will tend to move the pen off-axis rather than cause it to slide axially relative to the tube. The reaction force will therefore have a radial component that can be measured by a pressure sensor or a strain gauge.

Such a tool may imitate a hand when writing. The imitation may go as far as orientating the mount 14 in such a way that the pen is held at an angle from vertical to enable the wearer to clearly see what they are writing. The mount 14 may include an insert to accommodate different pen or pencil thicknesses. The insert should be made from a material which is firm enough to support the pen but has sufficient flexibility to accommodate different sizes. One example of a suitable material is a foam or sponge.

The mount 14 may be a simple connector for the tool to attach to. This simple connector mount is shown in FIG. 1. The tool 16 may also take the form of a pincer grip for holding additional tools as shown in FIG. 1.

The present invention is not limited to any specific type of mount or tool, provided the sensor of the present invention can be incorporated into the mount/tool configuration to enable feedback by providing instructions to the control circuit and the actuator.

Data transfer between the sensor 28 and the control circuit, and the control circuit and the actuator 20 may be through wires or contacts, or may instead be via a contactless link such as an inductive loop, electro-optical coupling, or wireless (e.g. Bluetooth®).

It will be appreciated that although the sensor described is a strain gauge or a pressure sensor, the disclosure encompasses embodiments where a sensor is used to measure any physical parameter as required by the nature of the tool.

It is also to be understood that although the drawings and the description relate to a prosthesis for an arm, the principles are equally applicable to a prosthesis for a leg.

Approximately as employed herein means ±10%.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

1. A prosthesis for an arm or a leg, comprising a flexible sleeve for receiving a residual limb, a constrictor on the flexible sleeve for tightening the sleeve around the residual limb, and a rigid mount secured to the sleeve and spaced axially along the sleeve from the constrictor for mounting a tool, wherein a sensor is provided in the mount to measure a physical parameter associated with the function performed by the tool and wherein the prosthesis further comprises an actuator connected to the constrictor and a control circuit acting on the actuator to vary the force tightening the sleeve around the residual limb in dependence upon the measured parameter.

2. A prosthesis for an arm or a leg, comprising a flexible sleeve for receiving a residual limb, a constrictor on the flexible sleeve for tightening the sleeve around the residual limb, a rigid mount secured to the sleeve and spaced axially along the sleeve from the constrictor and a mount for releasably attaching to the prosthesis a tool that incorporates a sensor for measuring a physical parameter associated with the function performed by the tool, wherein the prosthesis comprises an actuator connected to the constrictor and a control circuit communicating with the sensor by way of the mount and acting on the actuator to vary the force tightening the sleeve around the residual limb in dependence upon the measured parameter.

3. A prosthesis as claimed in claim 2, wherein the control circuit communicates with the sensor by way of electrical contacts in the mount and on the tool.

4. A prosthesis as claimed in claim 2, wherein the control circuit communicates with the sensor by a contactless link selected from an inductive loop, a photoelectric coupler, or wireless.

5. A prosthesis as claimed in claim 1, wherein the sensor is a pressure sensor or a strain gauge.

6. A prosthesis as claimed in claim 1, wherein the actuator is an electric motor driving an output shaft about which an elongate flexible member, serving as at least part of the constrictor, is wound.

7. A prosthesis as claimed in claim 6, in which the electric motor is connected to the output shaft by way of step-down gearing.

8. A prosthesis as claimed in claim 6, wherein an encoder is fitted to the output shaft to provide to the control circuit a feedback signal indicative of the angular position of the output shaft.

9. A method of providing the wearer of a prosthesis that grips a residual limb of the wearer with feedback regarding a function of a tool mounted on the prosthesis, in which method the gripping force applied by the prosthesis to the residual limb is varied in dependence upon a sensed physical parameter associated with the function performed by the tool.
